(19) United States
(12) Patent Application Publication
(10) Pub. No.: US 2024/0269084 A1
(43) Pub. Date: Aug. 15, 2024

(54) DELAYED RELEASE COMPOSITIONS OF DIMETHYL FUMARATE

(71) Applicant: Zim Laboratories Limited, Kalmeshwar, Nagpur (IN)

(72) Inventors: Chandrashekhar Mainde, Kalmeshwar, Nagpur (IN); Uttam Kedar, Kalmeshwar, Nagpur (IN); Kuldeep Gangawat, Kalmeshwar, Nagpur (IN)

(73) Assignee: Zim Laboratories Limited, Kalmeshwar, Nagpur (IN)

(21) Appl. No.: 18/566,965

(22) PCT Filed: Jun. 1, 2022
(86) PCT No.: PCT/IB2022/055120

§ 371 Date: Dec. 4, 2023

(30) Foreign Application Priority Data
Jun. 4, 2021 (IN) 202121024862
Jun. 4, 2021 (IN) 202121024863

(51) Int. Cl. A61K 9/50; A61K 31/225

(52) U.S. Cl. CPC A61K 9/5078; A61K 9/5015; A61K 9/5026; A61K 9/5047; A61K 31/225

(57) ABSTRACT

The present invention relates to delayed release pharmaceutical compositions comprising dimethyl fumarate in the treatment of multiple sclerosis. The delayed release composition of dimethyl fumarate is having particle size (D90) of dimethyl fumarate in the composition is less than 50 μm and composition is manufactured by extrusion-spheronization or layering method.

### CASE REFERENCE

The present application has been made combining two Indian application Nos. 202121024863 dated Jun. 4, 2021 and 202121024862 dated Jun. 4, 2021

### FIELD OF THE INVENTION

The present invention relates to delayed release compositions of dimethyl fumarate and process of manufacturing in the treatment of multiple sclerosis. Specifically the present invention relates to delayed release composition of dimethyl fumarate with particle size (D90) of dimethyl fumarate is less than 50 μm and process of manufacturing the composition by extrusion-spheronization or layering method.

### BACKGROUND OF THE INVENTION

Multiple sclerosis (MS) is a potentially disabling disease of the brain and spinal cord. In Multiple sclerosis, the immune system attacks the protective sheath (myelin) that covers nerve fibers and causes communication problems between your brain and the rest of your body. Eventually, the disease can cause permanent damage or deterioration of the nerves. The cause of MS is unknown; however, it is believed to occur as a result of some combination of genetic and environmental factors such as infectious agents. Signs and symptoms of MS vary widely and depend on the amount of nerve damage and which nerves are affected. Multiple sclerosis signs and symptoms may differ greatly from person to person and over the course of the disease depending on the location of affected nerve fibers. Symptoms often affect movement, such as Numbness or weakness in one or more limbs that typically occurs on one side of your body at a time or your legs and trunk, electric-shock sensations that occur with certain neck movements, especially bending the neck forward, Tremor, lack of coordination or unsteady gait. Vision problems are also common, including Partial or complete loss of vision, usually in one eye at a time often with pain during eye movement, prolonged double vision, Blurry vision. Multiple sclerosis symptoms may also include Slurred speech, Fatigue, Dizziness, Tingling or pain in parts of your body, Problems with sexual, bowel and bladder function. There is no cure for multiple sclerosis. Treatment typically focuses on speeding recovery from attacks, slowing the progression of the disease and managing MS symptoms. Treatment options for relapsing-remitting Multiple sclerosis include oral medications and injectable. Oral treatments include use of medications like Dimethyl fumarate, Fingolimod, Diroximel fumarate, Teriflunomide, Siponimod and Cladribine. The injectable medications include use of medications like Interferon beta and Glatiramer acetate

Dimethyl fumarate is known by its chemical name as dimethyl (E) butenedioate. Dimethyl fumarate having molecular mass of 144.13 and molecular weight of C₆H₈O₄. Dimethyl fumarate is represented by compound of structural formula I.

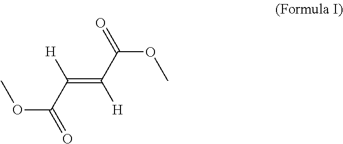

Dimethyl fumarate is a white to off-white powder that is highly soluble in water.

Dimethyl fumarate capsule was approved in USA on Mar. 27, 2013 under the trade name TECFIDERA® and is available in the strength of 120 mg and 240 mg. The product is indicated for the treatment of relapsing forms of multiple sclerosis (MS), to include clinically isolated syndrome, relapsing-remitting disease and active secondary progressive disease in adults.

Dimethyl fumarate under the tradename TECFIDERA® contain dimethyl fumarate as the active ingredient and inactive ingredients as microcrystalline cellulose, silicified microcrystalline cellulose, croscarmellose sodium, talc, silica colloidal silicon dioxide, magnesium stearate, triethyl citrate, methacrylic acid copolymer—Type A, methacrylic acid copolymer dispersion, simethicone (30% emulsion), sodium lauryl sulphate and polysorbate 80. The capsule shell, printed with black ink, contains the following inactive ingredients: gelatin, titanium dioxide, FD&C blue 1; brilliant blue FCF, yellow iron oxide and black iron oxide.

U.S. Pat. No. 7,619,001 discloses a method of treating multiple sclerosis comprising administering, to a patient in need of treatment for multiple sclerosis, an amount of a pharmaceutical preparation effective for treating multiple sclerosis, the pharmaceutical preparation comprising at least one excipient or at least one carrier or at least one combination thereof; and dimethyl fumarate, methyl hydrogen fumarate, or a combination thereof. Further, U.S. Pat. No. 7,619,001 discloses preparations of dimethyl fumarate in the form of micro-tablets or micro-pellets.

U.S. Pat. No. 8,399,514 discloses a method of treating a subject in need of treatment for multiple sclerosis comprising orally administering to the subject in need thereof a pharmaceutical composition consisting essentially of (a) a therapeutically effective amount of dimethyl fumarate, monomethyl fumarate or a combination thereof, and (b) one or more pharmaceutically acceptable excipients, wherein the therapeutically effective amount of dimethyl fumarate, monomethyl fumarate or a combination thereof is about 480 mg per day.

WO2016205270 discloses Dimethyl fumarate (DMF) particles having a volume median diameter (D50) between 50 μm and 100 μm.

Dimethyl fumarate sublimates at low temperature. Therefore, most of Dimethyl fumarate lost during manufacturing process and production. Also during long-term storage of dimethyl fumarate product, sublimation occur; therefore, which affects the potency of drug in the treatment of multiple sclerosis. Further handling of dimethyl fumarate is difficult since it causes eye irritation, skin irritation and damage due to its physical contact.

The commercial available product and product known in the prior art for dimethyl fumarate are available in the form of micro tablet, pellet, powder, granule and these products are prepared by the conventional methods. The conventional method involve operations like milling, compression. Due to this conventional methods and operations, more loss of drug by sublimation during manufacturing process and during shelf life of product. In addition, this conventional methods and operations involve more physical contact or exposure of drug during manufacturing process which causes eye irritation and skin damage. The product made in the prior art with conventional manufacturing process and operations suffers from drawbacks like dose dumping, poor dissolution profile, unstability and not having content uniformity; therefore lacks patient compliance in the treatment of multiple sclerosis.

Also, the product known in the prior art for dimethyl fumarate compositions are available with greater particle size which results in the poor dissolution and absorption of drug in the treatment of multiple sclerosis.

Therefore, there is need in the art to prepare pharmaceutical composition of dimethyl fumarate with smaller particle size, wherein manufacturing process minimizes sublimation of dimethyl fumarate during manufacturing process and during shelf life of product, process that minimizes physical contact of dimethyl fumarate, also the process that provide product with better stability, content uniformity, dissolution profile and which avoid dose dumping.

Accordingly applicant of the present invention invented, pharmaceutical composition of dimethyl fumarate; wherein manufacturing process minimizes sublimation, physical contact of drug and provides product with better stability, content uniformity, dissolution profile and which avoid dose dumping in the treatment of multiple sclerosis.

### OBJECT OF THE INVENTION

Accordingly, it is an object of this invention is to provide delayed release pharmaceutical composition of dimethyl fumarate having particle size (D90) of dimethyl fumarate which is less than 50 μm in the treatment of multiple sclerosis.

Another object of this invention is to provide process of manufacturing delayed release pharmaceutical composition of dimethyl fumarate by extrusion-spheronization method and having particle size (D90) of dimethyl fumarate less than 50 μm in the composition.

A further object of this invention is to provide process of manufacturing delayed release pharmaceutical composition of dimethyl fumarate by layering method and having particle size (D90) of dimethyl fumarate less than 50 μm in the composition.

A further object of this invention is to provide process of manufacturing delayed release pharmaceutical composition of dimethyl fumarate by extrusion-spheronization or layering method; wherein manufacturing process minimizes sublimation, physical contact of drug and provide product with better stability in the treatment of multiple sclerosis.

A further object of this invention is to provide delayed release pharmaceutical composition of dimethyl fumarate with (D90) less than 50 μm with optimum dissolution and which avoid dose dumping in the treatment of multiple sclerosis.

A further object of this invention is to provide method of treatment of multiple sclerosis involving administration of delayed release composition of dimethyl fumarate with (D90) less than 50 μm and which is manufactured by either extrusion-spheronization or layering method.

### SUMMARY OF THE INVENTION

A first aspect of the present invention is to provide delayed release pharmaceutical composition of dimethyl fumarate; wherein particle size (D90) of dimethyl fumarate is less than 50 μm.

In another aspect of the present invention is to provide delayed release pharmaceutical composition of dimethyl fumarate along with one or more excipient; wherein particle size (D90) of dimethyl fumarate is less than 50 μm.

In another aspect of the present invention is to provide delayed release pellets of dimethyl fumarate; wherein particle size (D90) of dimethyl fumarate is less than 50 μm.

In another aspect of the present invention is to provide delayed release pellets of dimethyl fumarate along with or more excipient; wherein particle size (D90) of dimethyl fumarate is less than 50 μm.

In another aspect of the present invention is to provide delayed release pharmaceutical composition of dimethyl fumarate comprising core, seal coating and enteric coating; wherein core is manufactured by either extrusion-spheronization or layering method.

In another aspect of the present invention is to provide delayed release pharmaceutical composition of dimethyl fumarate comprising core, seal coating and enteric coating; wherein core is manufactured by either extrusion or layering method and particle size (D90) of dimethyl fumarate in the composition is less than 50 μm.

In another aspect of the present invention is to provide delayed release pharmaceutical composition of dimethyl fumarate; wherein process of manufacturing involves steps of
i) Mixing dimethyl fumarate with one or more pharmaceutically acceptable excipient and granulating same with one or more solvent.
ii) Extrude the wet granulated mass in step i by extruder and Spheronize the same in spheronizer to form pellet.
iii) Coat the pellets by using seal coating solution.
iv) Coat the seal coated pellet by using delayed release coating.

In another aspect of the present invention is to provide delayed release pharmaceutical composition of dimethyl fumarate; wherein particle size (D90) of dimethyl fumarate is less than 50 μm and which process of manufacturing involves steps of
i) Mixing dimethyl fumarate with one or more pharmaceutically acceptable excipient and granulating same with one or more solvent.
ii) Extrudate the wet granulated mass in step i by extruder and Spheronize the same in spheronizer to form pellet.
iii) Coat the pellets by using seal coating solution.
iv) Coat the seal coated pellet by using delayed release coating.

In another aspect of the present invention is to provide delayed release pharmaceutical composition of dimethyl fumarate; wherein process of manufacturing involves steps of
i) Dispersing or dissolving Dimethyl fumarate in one or more pharmaceutically acceptable excipient, solvent; coating the said solution on the inert core.
ii) Coating the drug loaded core formed in step i with one or more pharmaceutically acceptable excipient and solvent to form seal coating.
iii) Coating the seal coated core formed in step ii with enteric polymer along with one or more pharmaceutically acceptable excipient and solvents.

In another aspect of the present invention is to provide delayed release pharmaceutical composition of dimethyl fumarate; wherein particle size (D90) of dimethyl fumarate is less than 50 μm and which process of manufacturing involves steps of
i) Dispersing or dissolving Dimethyl fumarate in one or more pharmaceutically acceptable excipient, solvent; coating the said solution on the inert core.
ii) Coating the drug loaded core formed in step i with one or more pharmaceutically acceptable excipient and solvent to form seal coating.
iii) Coating the seal coated core formed in step ii with enteric polymer along with one or more pharmaceutically acceptable excipient and solvents.

In another aspect of the present invention is to provide delayed release pharmaceutical composition of dimethyl fumarate along with one or more excipient; wherein particle size (D90) of dimethyl fumarate is less than 50 μm in the treatment of multiple sclerosis.

### DETAIL DESCRIPTION OF THE INVENTION

The present invention relates to delayed release compositions of dimethyl fumarate and process of manufacturing in the treatment of multiple sclerosis.

The delayed release compositions of dimethyl fumarate according to present invention comprises particle size (D90) of dimethyl fumarate which is less than 50 μm.

Particle size distribution means the cumulative volume size distribution as tested by any conventionally accepted method such as the laser diffraction method (i.e. Malvern analysis). The term (D10) means the size at which 10% by volume of the particles are finer. The term (D50) means the size at which 50% by volume of the particles are finer. The term (D90) means the size at which 90% by volume of the particles are finer. The particle size of dimethyl fumarate has been measured according to present invention by Malvern Laser Diffraction.

In another aspect of the present invention is to provide delayed release pharmaceutical composition of dimethyl fumarate along with one or more excipient; wherein particle size (D90) of dimethyl fumarate is less than 50 μm.

The delayed release pharmaceutical composition according to present invention contain active ingredient dimethyl fumarate in the range from 10 mg to 300 mg; preferably, the amount of dimethyl fumarate in the delayed release pharmaceutical composition according to present invention is 120 mg and 240 mg.

The one or more pharmaceutically acceptable excipient according to present invention is selected from the group consisting of Diluent, Adsorbent, Binder, Plasticizer, solvent, Anti-tacking Agent, seal coating polymer, delayed release polymer and solubilizing agent.

The diluent includes but not limited to Microcrystalline Cellulose, Mannitol, Lactose Anhydrous, Lactose Monohydrate, Ammonium Alginate, Calcium Carbonate, Calcium Lactate, Anhydrous Dibasic Calcium Phosphate, Dibasic Calcium Phosphate Dihydrate, Tribasic Calcium Phosphate, Calcium Sulfate, Corn Starch, Pregelatinized Starch, Dextrates, Dextrin, Dextrose, Erythritol, Fructose, Glyceryl Palmitostearate, Kaolin, Lactitol, Maltitol, Magnesium Carbonate, Magnesium Oxide, Maltodextrin, Maltose, Polydextrose, Polymethacrylates, Simethicone, Sodium Chloride, Sorbitol, Starch, Pregelatinized Starch, Sucrose, Sulfobutylether b-Cyclodextrin, Talc, Trehalose, Xylitol or combination thereof and alike. Preferably diluents is Microcrystalline Cellulose, Mannitol or combination thereof. The composition according to present invention contains diluent from 15 to 70% by weight of composition.

The adsorbent includes but not limited to Colloidal Anhydrous Silica, Aluminum Hydroxide Adjuvant, Aluminum Oxide, Attapulgite, Bentonite, Calcium Silicate, Microcrystalline Cellulose, Powdered Cellulose, Hectorite, Kaolin, Magnesium Aluminum Silicate, Magnesium Carbonate, Pectin, Polycarbophil, Saponite or combination thereof and alike. Preferably adsorbent is Colloidal Anhydrous Silica. The composition according to present invention contains Adsorbent from 0.05 to 10% by weight of composition.

The binder includes but not limited to Povidone, Acacia, Agar, Calcium Carbonate, Tribasic Calcium Phosphate, Carbomer, Carboxymethylcellulose Calcium, Microcrystalline Cellulose, Powdered Cellulose, Ceratonia, Chitosan, Dextrin, Ethylcellulose, Gelatin, Liquid Glucose, Guar Gum, Hydroxyethyl Cellulose, Hydroxyethylmethyl Cellulose, Hypromellose, Inulin, Lactose Monohydrate, Magnesium Aluminum Silicate, Maltodextrin, Methylcellulose, Polycarbophil, Polydextrose, Polyethylene Oxide, Polymethacrylates, Sodium Alginate, Pregelatinized Starch, Starch, Sucrose, Sunflower Oil, Hydrogenated Vegetable Oil, Vitamin E Polyethylene Glycol Succinate, Zein or combination thereof and alike. Preferably binder is Povidone. The composition according to present invention contains binder from 2 to 20% by weight of composition.

The Plasticizer includes but not limited to Triethyl Citrate, Benzyl Benzoate, Chlorobutanol, Dibutyl Sebacate, Diethyl Phthalate, Dimethyl Phthalate, Glycerin, Mannitol, Polyethylene Glycol, Propylene Glycol, Pyrrolidone, Sorbitol, Triacetin, Tributyl Citrate or combination thereof and alike. Preferably Plasticizer is Triethyl Citrate. The composition according to present invention contains Plasticizer from 0.05 to 5% by weight of composition.

The solvent include but not limited to isopropyl alcohol, dichloromethane, water, ethanol, acetone, methanol, acetone, Tetrachloroethylene, Toluene, Methyl acetate, Ethyl acetate or combination thereof and alike. Preferably solvent is isopropyl alcohol, dichloromethane and water or combination thereof.

The Anti-tacking Agent includes but not limited to purified talc, silicon dioxide, magnesium Stearate, stearic acid, glyceryl monostearate, sodium stearyl fumerate, sodium stearate or combination thereof and alike. Preferably Anti-tacking Agent is purified talc, silicon dioxide or combination thereof. The composition according to present invention contains Anti-tacking Agent from 0.05 to 10% by weight of composition.

The seal coating polymer includes but not limited to hydroxyl propyl methyl cellulose, hydroxyl propyl cellulose, methylcellulose, ethyl cellulose or combination thereof and alike. Preferably seal coating polymer is hydroxyl propyl methyl cellulose. The composition according to present invention contains seal coating polymer from 2 to 30% by weight of composition.

The delayed release polymer includes but not limited to Methacrylic Acid-Ethyl Acrylate Copolymer, Methyl acrylate-methacrylic acid copolymers, Cellulose acetate phthalate (CAP), Cellulose acetate succinate, Hydroxypropyl methyl cellulose phthalate, Hydroxypropyl methyl cellulose acetate succinate (hypromellose acetate succinate), Polyvinyl acetate phthalate (PVAP), Methyl methacrylate-methacrylic acid copolymers, Shellac or combination thereof and alike. Preferably delayed release polymer is Methacrylic Acid-Ethyl Acrylate Copolymer. The composition according to present invention contains delayed release polymer from 5 to 50% by weight of composition.

The solubilizing agent includes but not limited to Benzalkonium Chloride, Benzyl Benzoate, Cetylpyridinium Chloride, Glyceryl Monostearate, Hypromellose, Macrogol 15 Hydroxystearate, Lecithin, Hydroxypropyl Betadex, Cyclodextrins, Phospholipids, Polyoxyethylene Alkyl Ethers, Polyoxyethylene Castor Oil Derivatives, Polyoxyethylene Sorbitan Fatty Acid Esters, Polyoxyethylene Stearates, Polyoxylglycerides, Pyrrolidone, Sorbitan Fatty Acid Esters, Stearic Acid, Sulfobutylether b-Cyclodextrin, Triolein and Vitamin E Polyethylene Glycol Succinate or combination thereof and alike.

The delayed release pharmaceutical composition of dimethyl fumarate according to present invention may be in the form of micro granule, granule, pellet, bead, spheroid, capsule and tablet.

In another aspect of the present invention is to provide delayed release pharmaceutical composition of dimethyl fumarate comprising core, seal coating and enteric coating; wherein core is manufactured by either extrusion-spheronization or layering method.

The core of the delayed release pharmaceutical composition according to present invention may be manufactured by either extrusion-spheronization or layering method.

An extrusion-spheronization method according to present invention involves, first the dry powder mix is agglomerated with the help of a binding liquid. Then it is processed in the extruder to produce high-density extrudates. These extrudates are finally converted to pellets on spheronizer.

The layering method of core formation according to present invention involves deposition of successive layers of drug and binder solution/suspension on started seeds which can be either an inert material or granules of same drug.

In another aspect of the present invention is to provide delayed release pharmaceutical composition of dimethyl fumarate; wherein process of manufacturing involves steps of
i) Mixing dimethyl fumarate with one or more pharmaceutically acceptable excipient and granulating same with one or more solvent.
ii) Extrude the wet granulated mass in step i by extruder and Spheronize the same in spheronizer to form pellet.
iii) Coat the pellets by using seal coating solution.
iv) Coat the seal coated pellet by using delayed release coating.

In another embodiment of the present invention is to provide delayed release pharmaceutical composition of dimethyl fumarate; wherein process of manufacturing involves steps of
i) Mixing dimethyl fumarate with one or more pharmaceutically acceptable excipient.
ii) Preparing binder solution by mixing binder with one or more solvent or mixture thereof.
iii) Granulating mixture in step i with binder solution of step ii.
iv) Extrude the wet material formed in step iii in extruder.
v) Spheronize the extrudes formed in step iv, to form spherical pellets.
vi) Coat the pellets by using coating solution comprising excipient and solvent to form seal coating.
vii) Coat the seal coated pellet by using delayed release polymer to form enteric-coated dimethyl fumarate pellet.

In another embodiment of the present invention is to provide delayed release pharmaceutical composition of dimethyl fumarate; wherein particle size (D90) of dimethyl fumarate is less than 50 μm and which process of manufacturing involves steps of
i) Mixing dimethyl fumarate with one or more pharmaceutically acceptable excipient and granulating same with one or more solvent.
ii) Extrude the wet granulated mass in step i by extruder and Spheronize the same in spheronizer to form pellet.
iii) Coat the pellets by using seal coating solution.
iv) Coat the seal coated pellet by using delayed release coating.

In another embodiment of the present invention is to provide delayed release pharmaceutical composition of dimethyl fumarate; wherein particle size (D90) of dimethyl fumarate is less than 50 μm and which process of manufacturing involves steps of
i) Mixing dimethyl fumarate with one or more pharmaceutically acceptable excipient.
ii) Preparing binder solution by mixing binder with one or more solvent or mixture thereof.
iii) Granulating mixture in step i with binder solution of step ii formed with one or more solvent or mixture thereof.
iv) Extrude the wet material formed in step iii in extruder.
v) Spheronize the extrudes formed in step iv to form spherical pellets.
vi) Coat the pellets by using coating solution comprising excipient and solvent to form seal coating.
vii) Coat the seal coated pellet by using delayed release polymer to form enteric-coated dimethyl fumarate pellet.

In another aspect of the present invention is to provide delayed release pharmaceutical composition of dimethyl fumarate; wherein process of manufacturing involves steps of
i) Dispersing or dissolving Dimethyl fumarate in one or more pharmaceutically acceptable excipient, solvent; coating the said solution on the inert core.
ii) Coating the drug loaded core formed in step i with one or more pharmaceutically acceptable excipient and solvent to form seal coating.
iii) Coating the seal coated core formed in step ii with enteric polymer along with one or more pharmaceutically acceptable excipient and solvents.

In another embodiment of the present invention is to provide delayed release pharmaceutical composition of dimethyl fumarate; wherein particle size (D90) of dimethyl fumarate is less than 50 μm and which process of manufacturing involves steps of
i) Dispersing or dissolving Dimethyl fumarate in one or more pharmaceutically acceptable excipient, solvent; coating the said solution on the inert core.
ii) Coating the drug loaded core formed in step i with one or more pharmaceutically acceptable excipient and solvent to form seal coating.
iii) Coating the seal coated core formed in step ii with enteric polymer along with one or more pharmaceutically acceptable excipient and solvents.

The amount of dimethyl fumarate, excipients in the composition and manufacturing process i.e. either by extrusion-spheronization or layering method has been optimized in such way that, delayed release composition according to present invention minimizes sublimation, physical contact of dimethyl fumarate and provides product with better stability, content uniformity, dissolution profile and avoid dose dumping.

The delayed release pharmaceutical composition of dimethyl fumarate with (D90) less than 50 μm and which is manufactured by either extrusion-spheronization or layering method provides optimum dissolution of the product required in the treatment of multiple sclerosis. The dissolution of the test product according to present invention and reference product Tecfidera 240 mg has been performed in 0.1N Hydrochloride Acid followed by pH 6.8 Phosphate Buffer; USP Type 2 Paddle with Sinker apparatus 100 rpm. The results of dissolution of test product have been found to be in compliance with that of reference product.

The delayed release composition of dimethyl fumarate according to present invention in the form of pellet, granule, bead, spheroid, capsule or tablet can be packaged in suitable airtight containers and moisture proof packs. Packaging may include but not limited to high-density polyethylene bottle, aluminum blister package. The container material or packaging material of the present invention does not affect the quality of the preparation or does not allow diffusion of any kind into or across the material of the container into the preparation.

### EXAMPLES

Some illustrative non-limiting examples of the present invention are described below

TABLE 1
Example 1
Extrusion-spheronization method

| | | |
|---|---|---|
| Sr. No. | Ingredients | mg/Capsule |

Dry Mixing

| | | |
|---|---|---|
| 01 | Dimethyl Fumarate | 240.000 |
| 02 | Microcrystalline Cellulose | 22.000 |
| 03 | Mannitol | 84.000 |
| 04 | Colloidal Anhydrous Silica (Aerosil) | 10.000 |

Binder Preparation

| | | |
|---|---|---|
| 05 | Povidone (PVP K-30) | 30.000 |
| 06 | Purified Water | Os |
| 07 | Isopropyl Alcohol | Qs |

Seal Coating

| | | |
|---|---|---|
| 08 | Hydroxypropyl Methylcellulose (HPMC 5 cps) | 15.000 |
| 09 | Triethyl Citrate | 4.000 |
| 10 | Isopropyl Alcohol | Qs |
| 11 | Dichloromethane | Qs |

Delayed Release Coating

| | | |
|---|---|---|
| 12 | Methacrylic Acid - Ethyl Acrylate Copolymer | 72.000 |
| | (1:1) Dispersion 30% (Eudragit L30 D55) | |
| 13 | Triethyl Citrate | 7.000 |
| 14 | Purified Talc | 3.000 |
| 15 | Purified water | Qs |

| | |
|---|---|
| Total Weight | 487.000 |

### Manufacturing Process:

### Stage I: Raw Material Sifting

Co-sift the dimethyl fumarate, microcrystalline cellulose, mannitol and colloidal anhydrous silica using appropriate sieve.

Collect co-sifted materials in double polythene lined HDPE container having stage label as co-sifted material ready for Dry Mixing.

### Stage II Binder Preparation

Take required quantity of Purified water in SS vessel and add Povidone PVP K-30 under continuous stirring to get clear solution. Then add Isopropyl alcohol into it and used as binder solution.

### Stage III: Dry Mixing

Load co-sifted materials in Rapid mixer granulator (RMG) and carry out dry mixing for suitable time at suitable impeller speed and chopper off.

### Stage IV: Granulation

Granulate the dry mixed material using binder solution of stage II, at suitable impeller and chopper speed for suitable time.

### Stage V: Extrusion

Load the wet material for extrusion and extrude the wet material at the suitable speed using suitable screen.

### Stage VI: Spheronization

Spheronize the extrudes at suitable speed for suitable time to get desired spherical pellets.

### Stage VII: Drying and Sifting

Dry the wet pellets in Fluidized bed coater (FBC) at suitable temperature for suitable time to achieve target LOD and sift the dried pellets using suitable mesh ASTM.

### Stage VIII: Seal Coating

Preparation of Seal Coating solution using Hydroxypropyl methylcellulose (HPMC 5 cps) & Triethyl citrate with Isopropyl Alcohol and Dichloromethane.

Spray the seal coating solution on dried Pellets in FBC to get desired weight gain.

Dry the pellets in Fluidized bed coater (FBC) and sift through suitable mesh ASTM.

### Stage IX: Delayed Release Coating

Preparation of coating solution using Methacrylic Acid-Ethyl Acrylate Copolymer (1:1) Dispersion 30% (Eudragit L 30 D 55), Talc and Triethyl citrate with Purified water.

Spray the coating solution on Seal coated pellets in FBC to get desired weight gain.

Dry the pellets in FBC and sift through suitable mesh ASTM.

### Dissolution Profile for Example 1

Test Product (Tₜ): Dimethyl Fumarate DR Capsules 240 mg as per example 1

Reference Product (Rₜ): Tecfidera 240 mg

Dissolution Medium: 0.1N Hydrochloride Acid followed by pH 6.8 Phosphate Buffer; USP Type 2 (Paddle with Sinker), 100 rpm.

TABLE 2
Dissolution profile for Example 1:

Average % drug release

| | | | |
|---|---|---|---|
| Sr. | | Rₜ | Tₜ |
| No. | Time Interval (Min) | (Reference) | (Test) |

Acid stage

| | | | |
|---|---|---|---|
| 1 | 120 | 1 | 2 |

Buffer Stage

| | | | |
|---|---|---|---|
| 1 | 10 | 86 | 70 |
| 2 | 15 | 89 | 82 |
| 3 | 20 | 90 | 89 |
| 4 | 30 | 89 | 92 |
| 5 | 45 | 89 | 93 |
| 6 | 60 | 89 | 93 |

| | |
|---|---|
| F2 Value | 70 |

Conclusion: As per above result, the dissolution of the test product as per example 1, were found to be similar to reference product Tecfidera 240 mg.

TABLE 3
Example 2
Layering Method

| | | |
|---|---|---|
| | | Quantity in |
| Sr. No. | Ingredients | mg/Capsule |

Drug Layering

| | | |
|---|---|---|
| 1 | Dimethyl Fumarate | 240.000 |
| 2 | Sugar Globules 30/40# | 117.500 |
| 3 | Hydroxy propyl methyl cellulose 5 cps | 10.000 |
| 4 | Povidone PVP K-30 | 40.000 |
| 5 | Triethyl Citrate | 2.000 |
| 6 | Polysorbate 80 | 5.000 |
| 7 | Isopropyl alcohol | QS |
| 8 | Dichloromethane | QS |

Seal Coating

| | | |
|---|---|---|
| 9 | Hydroxy propyl methyl cellulose 5 cps | 6.000 |
| 10 | Triethyl Citrate | 1.500 |
| 11 | Isopropyl alcohol | QS |
| 12 | Dichloromethane | QS |

Delayed Release Coating

| | | |
|---|---|---|
| 13 | Methacrylic acid - Methyl Methacrylate | 40.000 |
| | Copolymer (1:1) | |
| 14 | Triethyl citrate | 8.000 |
| 15 | Polysorbate 80 | 2.000 |
| 16 | Purified Talc | 8.000 |
| 17 | Isopropyl alcohol | QS |
| 18 | Dichloromethane | QS |

| | |
|---|---|
| Total Weight | 480.000 |

### Manufacturing Process

### Stage I: Drug Layering

Disperse Dimethyl Fumarate in Dichloromethane. On the other hand Disperse Povidone PVP K-30 in Isopropyl alcohol followed by Polysorbate 80 and Triethyl citrate, to this solution add drug solution and stir till solution becomes homogeneous and coat this solution on Sugar globules 30/40 #.

### Stage II: Seal Coating

Load drug layered core micro granules in FLP/FBC/FBP and Disperse Hydroxy propyl methyl cellulose 5cps in Isopropyl alcohol and add Dichloromethane under continuous stirring followed by Triethyl citrate with the help of lab stirrer and coat on drug loaded core microgranules.

### Stage III: Delayed Release Coating

Load sub coated pellets in FLP/FBC/FBP and Disperse Methacrylic acid-Methyl Methacrylate Copolymer (1:1) in Isopropyl alcohol and add Dichloromethane under continuous stirring followed by Triethyl citrate with the help of lab stirrer then add Polysorbate 80 followed by purified talc and stir till solution becomes homogeneous and coat on sub coated pellets.

### Dissolution Profile for Example 2

Test Product (Tₜ): Dimethyl Fumarate DR Capsules 240 mg as per example 2

Reference Product (Rₜ): Tecfidera 240 mg

Dissolution Medium: 0.1N Hydrochloride Acid followed by pH 6.8 Phosphate Buffer; USP Type 2 (Paddle with Sinker), 100 rpm.

TABLE 4
Dissolution profile for Example 2:

Average % drug release

| | | | |
|---|---|---|---|
| Sr. | | Rₜ | Tₜ |
| No. | Time Interval (Min) | (Reference) | (Test) |

Acid stage

| | | | |
|---|---|---|---|
| 1 | 120 | 1 | 4 |

Buffer Stage

| | | | |
|---|---|---|---|
| 1 | 10 | 86 | 86 |
| 2 | 15 | 89 | 87 |
| 3 | 20 | 90 | 87 |
| 4 | 30 | 89 | 86 |
| 5 | 45 | 89 | 85 |
| 6 | 60 | 89 | 85 |

| | |
|---|---|
| F2 Value | 77 |

Conclusion: As per above results, the dissolution of the test product as per example 2, were found to be similar to reference product Tecfidera 240 mg.

We claim:

1. A delayed release pharmaceutical composition of dimethyl fumarate along with one or more pharmaceutically acceptable excipient, which comprises
a) Core
b) Optionally seal coating and
c) Enteric coating;
Wherein core is manufactured by either extrusion spheronization or layering method.

2. The delayed release pharmaceutical composition as claimed in claim 1, wherein core is manufactured by extrusion-spheronization method which involves
a) Mixing dimethyl fumarate with one or more pharmaceutically acceptable excipient and granulating same with one or more solvent.
b) Extrude the wet granulated mass in step (a) by extruder and Spheronize the same in spheronizer to form pellet or core.

3. The delayed release pharmaceutical composition as claimed in claim 1, wherein core is manufactured by layering method which involves
a) Dispersing or dissolving Dimethyl fumarate in one or more pharmaceutically acceptable excipient and solvent.
b) Coating the solution in step (a) on the inert core to form the core.

4. The delayed release pharmaceutical composition as claimed in claim 1, wherein particle size (D90) of dimethyl fumarate is less than 50 μm in the composition.

5. The delayed release pharmaceutical composition as claimed in claim 1, wherein composition is in the form of pellet, granule, bead, spheroid, capsule, tablet or micro granule.

6. The delayed release pharmaceutical composition of dimethyl fumarate as claimed in claim 1, wherein amount of dimethyl fumarate is ranging from 10 mg to 300 mg.

7. The delayed release pharmaceutical composition of dimethyl fumarate as claimed in claim 1, wherein amount of dimethyl fumarate is 120 mg or 240 mg.

8. The delayed release pharmaceutical composition of dimethyl fumarate as claimed in claim 1, wherein one or more pharmaceutically acceptable excipient is selected from the group consisting of Diluent, Adsorbent, Binder, Plasticizer, solvent, Anti-tacking Agent, seal coating polymer, delayed release polymer and solubilizing agent.

9. The delayed release pharmaceutical composition of dimethyl fumarate as claimed in claim 8, wherein diluent is Microcrystalline Cellulose, Mannitol or mixture thereof; adsorbent is Colloidal Anhydrous Silica; binder is povidone; plasticizer is triethyl citrate; solvent are isopropyl alcohol, dichloromethane or water or mixture thereof; anti-tacking agent is talc or silicon dioxide or mixture thereof, seal coating polymer is hydroxyl propyl methyl cellulose and delayed release polymer is Methacrylic Acid-Ethyl Acrylate Copolymer.

10. The delayed release pharmaceutical composition of dimethyl fumarate as claimed in claim 1, for the treatment of multiple sclerosis.
